(19) United States
(12) Patent Application Publication
(10) Pub. No.: US 2023/0149908 A1
(43) Pub. Date: May 18, 2023

(54) CATALYST COMPOSITIONS AND METHODS OF PREPARATION AND USE THEREOF

(71) Applicants: BASF CORPORATION, Florham Park, NJ (US); THE REGENTS OF THE UNIVERSITY OF CALIFORNIA, Oakland, CA (US)

(72) Inventors: Joseph C. Dellamorte, Beachwood, OH (US); Enrique Iglesia, Berkeley, CA (US); Biswanath Dutta, Berkeley, CA (US); Miao Guang, Guangzhou (CN)

(21) Appl. No.: 17/913,512

(22) PCT Filed: Feb. 19, 2021
(86) PCT No.: PCT/US2021/018692

§ 371 Date: Sep. 22, 2022

Related U.S. Application Data
(60) Provisional application No. 62/993,878, filed on Mar. 24, 2020.

(51) Int. Cl. B01J 23/89; B01J 23/75; B01J 23/86; B01J 23/825; B01J 21/08; B01J 21/04; B01J 35/00; B01J 35/10; B01J 35/02; B01J 37/02; C07C 5/48; C07C 5/333

(52) U.S. Cl. CPC B01J 23/8913; B01J 23/75; B01J 23/864; B01J 23/825; B01J 21/08; B01J 21/04; B01J 35/0066; B01J 35/1019; B01J 35/026; B01J 37/0203; C07C 5/48; C07C 5/3332; C07C 2523/75; C07C 2523/89; C07C 2523/86; C07C 2523/825; C07C 2521/08; C07C 2521/04

(57) ABSTRACT

Disclosed are catalyst compositions containing cobalt II cations (Co2+) on a support. In embodiments, the catalyst compositions are free of chromium and/or a precious metal. Also disclosed are methods of preparing such catalyst compositions and methods of using such catalyst compositions, for example, to dehydrogenate light alkane and/or light alkene gas.

### FIELD

This disclosure relates generally to catalyst compositions, for example, containing cobalt. The disclosure also relates to methods of preparing and using such catalyst compositions for dehydrogenating light alkane gas (and/or light alkene gas).

### BACKGROUND

Light alkenes, such as propene and ethene, are essential feedstocks for preparing a vast array of chemicals, such as polymers (e.g., polyethylene), oxygenates (e.g., ethylene glycol, acetaldehyde, and acetone) and chemical intermediates (e.g., ethylbenzene and propionaldehyde). In 2016, the global alkene market was valued at $250 billion and is expected to increase at an annual rate of about 6% over the next five years. These valuable intermediates are predominantly produced by steam-cracking or fluid catalytic cracking of crude oils and its by-products. However, growing demand for various petrochemicals coupled with decreasing petroleum reserves have shifted focus toward producing alkenes via alternate, but efficient and economical, methods.

On-purpose dehydrogenation of alkanes (e.g., propane and isobutane) has gained attention as an alternate route of producing alkenes. Such dehydrogenation methods are often based on natural/shale gas feedstocks. These feedstocks contain significantly fewer impurities and a higher hydrogen to carbon (H/C) ratio than reactants derived from crude oil, which also makes these methods more environmentally friendly. Alkane dehydrogenation technology is expected to produce more than 2×10⁸ ton year⁻¹ of light alkenes globally by the end of 2020.

Conventionally, alkane dehydrogenation has been practiced using supported platinum (Pt)-based and chromium-based (e.g., CrOₓ) catalysts. Two industrial processes for the production of light alkene using dehydrogenation are 1) the chromia-alumina-based Catofin® process; and 2) the Pt—Sn-based Oleflex™ process. However, the use of chromium (Cr) and platinum (Pt) based catalysts presents costs, environmental and health issues.

For example, platinum-based catalysts used for alkane dehydrogenation can include Pt deposited alone, or in combination with another material such as tin (Sn), on an inactive support. However, platinum-based catalysts are costly, sensitive to impurities, subject to deactivation and regenerable via a challenging regeneration process. The re-dispersion of Pt in spent catalysts often requires the addition of chlorine-based compounds during the catalyst regeneration process, which is ecologically harmful. Chromium-based catalysts containing hexavalent chromium (Cr(VI)) are toxic and present health issues. For example, according to the Occupational Safety and Health Administration (OSHA), human exposure to chromium (VI) may cause serious health issues such as lung cancer.

There is a need for methods and catalyst compositions that inhibit deactivation of the catalyst composition and at same time maintain a considerable dehydrogenation activity. There is a further need for catalyst compositions that are free of Cr and precious metals, such as Pt, and that increase the safety and sustainability of the dehydrogenation process. The use of such catalyst compositions in the dehydrogenation of light alkanes (and/or alkenes) could improve the total yield of dehydrogenation products within one cycle and require less frequent catalyst regenerations.

### BRIEF DESCRIPTION OF THE DRAWINGS

The present disclosure is illustrated by way of example, and not by way of limitation, in the figures of the accompanying drawings in which like references indicate similar elements. It should be noted that different references to an or one embodiment in this disclosure are not necessarily to the same embodiment, and such references mean at least one.

FIG. 1 shows an ultraviolet visible (UV-vis) spectra of 0.2 CoAl, 0.8 CoAl, 3.7 CoAl and 16 CoAl catalyst compositions according to embodiments.

FIG. 2a shows in-situ X-ray absorption spectra of a 0.8 CoSi catalyst composition according to embodiments.

FIG. 2b shows change of the K-edge intensity of Co as a function of time (sec) in H₂ at 873 K for 0.8 CoSi and 1.5 CoSi catalyst compositions according to embodiments.

FIG. 3a shows the effect of CoOₓ surface density of the rate (per mass) of a CoSi catalyst composition according to embodiments at reaction conditions: 873 K, 13.6 kPa PC₃H₈ and pre-treatment condition: 873 K, 101.3 kPa PHe, 0.5 h.

FIG. 3b shows the effect of CoOₓ surface density of the rate (per mass) of a CoAl catalyst composition according to embodiments at reaction conditions: 873 K, 13.6 kPa PC₃H₈ and pre-treatment condition: 873 K, 101.3 kPa PHe, 0.5 h.

FIG. 4a shows the effect of CoOₓ surface density of the rate (per Co) and selectivity of a CoSi catalyst composition at reaction conditions: 873 K, and 13.6 kPa PC₃H₈ and pre-treatment condition: 873 K, 101.3 kPa PHe, 0.5 h.

FIG. 4b shows the effect of CoOₓ surface density of the rate (per Co) and selectivity of a CoAl catalyst composition at reaction conditions: 873 K, and 13.6 kPa PC₃H₈ and pre-treatment condition: 873 K, 101.3 kPa PHe, 0.5 h.

FIG. 5a shows a comparison of the propane dehydrogenation rate (per mass) between Al₂O₃ and aluminum oxy-hydroxide supported Co-catalyst compositions according embodiments at reaction conditions: 873 K, and 13.6 kPa PC₃H₈ and pre-treatment condition: 873 K, 101.3 kPa PHe, 0.5 h.

FIG. 5b shows a comparison of propane dehydrogenation rate (per mass) between SiO₂ and silicon oxy-hydroxide supported Co-catalysts at reaction conditions: 873 K, and 13.6 kPa PC₃H₈ and pre-treatment condition: 873 K, 101.3 kPa PHe, 0.5 h.

FIG. 6a show infrared spectra of CO adsorbed on aluminum oxy-hydroxide supported CoOₓ catalyst compositions according to embodiments at 268-273 K (1.0 kPa CO, 99.0 kPa He) after treatment in flowing He (0.7 cm³ g⁻¹ s⁻¹) at 473 K for 1 h.

FIG. 6b show integrated CO adsorption peak areas for differently loaded aluminum oxy-hydroxide supported CoOₓ catalyst compositions according to embodiments measured at 268-273 K over the range 0.2-1 kPa CO.

FIG. 7 shows the propane dehydrogenation rate (per Co) as a function of CO-IR area at saturation (per Co) at 268-273 K for aluminum oxy-hydroxide supported CoOₓ catalyst composition according to embodiments, after treatment in flowing He (0.7 cm³ g⁻¹ s⁻¹) at 473 K for 1 h.

FIG. 8 shows a comparison of the propane dehydrogenation rate among different catalytic systems according to various embodiments herein.

FIG. 9 shows the change and stability of propane dehydrogenation rate (per mass) on 0.8 CoSi catalyst composition according to embodiments at reaction conditions: 873 K, 13.5 kPa PC₃H₈, 0-15.8 kPa PH₂ and pre-treatment condition: 873 K, 101.3 kPa PHe, 0.5 h.

FIG. 10 shows the effect of hydrogen (H₂) pressure on the propane dehydrogenation rate and hydrogenolysis on a 1.5 CoSi catalyst composition according to embodiments at reaction conditions: 873 K, 13.6 kPa PC₃H₈, 0-16 kPa PH₂; pre-treatment condition: 873 K, 101.3 kPa PHe, 0.5 h.

FIG. 11 shows the effect of hydrogen (H₂) pressure on the propane dehydrogenation rate on a 0.8 CoSi catalyst composition at reaction conditions: 873 K, 13.6 kPa PC₃H₈, 5-70 kPa PH₂ and pre-treatment condition: 873 K, 101.3 kPa PH₂, 12 h.

FIG. 12 shows the effect of propane (C₃H₈) pressure at the rate (per mass) of propane dehydrogenation reaction of 1.5 CoSi catalyst at reaction conditions: 873 K, 5-80 kPa PC₃H₈ and pre-treatment condition: 873 K, 101.3 kPa PH₂, 12 h.

FIG. 13 shows the effect of residence time (RT) on the rate (per mass) of propane dehydrogenation reaction of a 0.8 CoSi catalyst composition at reaction conditions: 873 K, 15 kPa H₂, and 22.5 kPa PC₃H₈ and pre-treatment condition: 873 K, 101.3 kPa PH₂, 12 h.

FIG. 14 shows the contribution of catalytic cracking reaction of propane on 1.5 CoSi catalyst composition to form methane and ethylene at reaction conditions: 873 K, 8.3-15 kPa PC₃H₈, 0 kPa PH₂ and pre-treatment condition: 873 K, 101.3 kPa PHe, 0.5 h.

FIG. 15 shows the effect of hydrogen (H₂) pressure on the rate ratio of methane and ethylene on a 1.5 CoSi catalyst composition at reaction conditions: 873 K, 8.3-15 kPa PC₃H₈, 0-60 kPa PH₂ and pre-treatment condition: 873 K, 101.3 kPa PHe, 0.5 h.

### BRIEF SUMMARY

According to embodiments, disclosed herein is a catalyst composition, comprising: a support comprising cobalt (II) (Co²⁺) cations, wherein the catalyst composition is free of at least one of chromium and a precious metal.

In further embodiments, disclosed herein is a method of preparing a catalyst composition, comprising: loading cobalt (II) (Co²⁺) cations onto a support, wherein the catalyst composition is free of at least one of chromium and a precious metal.

In yet further embodiments, disclosed herein is a method for dehydrogenating at least one of a light alkane gas and a light alkene gas, comprising: contacting the at least one light alkane gas and light alkene gas with a catalyst composition comprising a support comprising cobalt (II) cations (Co²⁺).

Further disclosed herein is a kit comprising: a catalyst composition according to embodiments herein; and instructions for using the catalyst composition according to embodiments herein.

### DETAILED DESCRIPTION

Described herein are methods of using catalyst compositions for dehydrogenating light alkane gases (and/or light alkene gases). Also disclosed are catalyst compositions for such dehydrogenation reactions and methods of preparation thereof. It is to be understood that the invention is not limited to the details of construction or process steps set forth in the following description. The invention is capable of other embodiments and of being practiced or being carried out in a variety of ways.

Reference throughout this specification to one embodiment, certain embodiments, one or more embodiments or an embodiment means that a particular feature, structure, material, or characteristic described in connection with the embodiment is included in at least one embodiment of the invention. Thus, the appearances of the phrases such as in one or more embodiments, in certain embodiments, in one embodiment or in an embodiment in various places throughout this specification are not necessarily referring to the same embodiment of the invention. Furthermore, the particular features, structures, materials, or characteristics may be combined in any suitable manner in one or more embodiments.

As used herein, the singular forms a, an, and the include plural references unless the context clearly indicates otherwise. Thus, for example, reference to a catalyst material includes a single catalyst material as well as a mixture of two or more different catalyst materials.

As used herein, the term about in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term about includes the recited number ±10%, such that about 10 would include from 9 to 11.

The term at least about in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term at least about includes the recited number minus 10% and any quantity that is higher such that at least about 10 would include 9 and anything greater than 9. This term can also be expressed as about 10 or more. Similarly, the term less than about typically includes the recited number plus 10% and any quantity that is lower such that less than about 10 would include 11 and anything less than 11. This term can also be expressed as about 10 or less.

Unless otherwise indicated, all parts and percentages are by weight except that all parts and percentages of gas are by volume. Weight percent (wt %), if not otherwise indicated, is based on an entire composition free of any volatiles, that is, based on dry solids content. Volume percent (vol %), if not otherwise indicated, is based on the total volume of the gas.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

Propane dehydrogenation (PDH) reactions can be conducted in the presence of Co-based catalysts on a support (e.g., SiO₂ and Al₂O₃ supports). The term “support” as used herein refers to a solid phase structure on which or into which a material is deposited or impregnated, respectively. Single-site Co²⁺ materials on silica supports (Co²⁺/SiO₂) have been formed using chloride (Cl) and an ammonia (NH₃)-based Co³⁺ precursor (Co(NH₃)₆Cl₃). Such materials have an initial PDH rate (per mass) of 12.8 mol·kg_{cat}⁻¹·h⁻¹ at 923 K, which upon deactivation over 20 h, decreases to a rate of 6.6 mol·kg_{cat}⁻¹·h⁻¹. Mesoporous Co—Al spinel catalysts have been formed by an intricate and complex procedure involving pluronic-123 (P123) as a soft template, and also may experience deactivation of the initial PDH rate (per mass) of 5.2 mol·kg_{cat}⁻¹·h⁻¹ to a PDH rate (per mass) of 1.1 mol·kg_{cat}⁻¹·h⁻¹ at 873 K after 5 h. Frequent regenerations under several oxidative conditions may destroy the well-defined mesoporous structure resulting from the templating surfactant. Prior Co-based catalysts for PDH reactions, have not improved the robustness and stability of the catalyst compositions and the catalysts have experienced rapid deactivation, for example, resulting in a half-life of 21 h for Co²⁺/SiO₂ and 2.5 h for Co—Al spinels, or from an initial PDH rate of 4 mol·kg_{cat}⁻¹·h⁻¹ to 1.6 mol·kg_{cat}⁻¹·h⁻¹ at 823 K after 10 h with a half-life of 7.5 h for a Co-catalyst supported on SiO₂.

According to embodiments, disclosed herein are catalyst compositions comprising Co²⁺ cations. The cations may be dispersed on a support. In embodiments, the catalyst compositions may be prepared via a low-cost, one-step synthesis procedure using earth-abundant non-toxic and non-corrosive reagents. The method prepares catalyst compositions having highly dispersed cobalt (II) (Co²⁺) cations, for example, in the form of cobalt (II) oxide, on a support. The resulting catalyst compositions are stable against further reduction under the high temperature and highly reducing conditions of an alkane dehydrogenation catalysis. The catalyst compositions are also efficient in the conversion of light alkanes to corresponding alkenes (or alkenes to butadienes). The catalyst compositions and structures can be designed for specific applications and alkane (or alkene) feedstocks.

In embodiments, the catalyst compositions convert light alkanes (e.g., propane) to alkenes (e.g., propene), or light alkenes to butadienes, with high selectivity and efficient reaction rates. According to embodiments, the dehydrogenation reaction is in the absence of oxygen.

In embodiments, the one-step synthesis may be a one-step grafting method that allows systematic variations of the surface density of active cobalt (Co) sites on a wide range of support materials that enable structure-activity relationships. This simple grafting method is well suited for scale-up and uses earth-abundant material. The grafting method allows systematic variations in the surface density of active sites on different supports as a tool to understand the nature and reactivity of the active sites and to develop an efficient and robust catalyst for dehydrogenation reactions. For example, a catalyst composition formed by the grafting method can have cobalt (II) (Co²⁺) cations on a silicon dioxide (SiO₂) support. In embodiments, the resulting catalyst composition has a cobalt surface density of 0.4 atoms nm⁻² on SiO₂ and provides a highly-stable propane dehydrogenation rate of 10 mol·Kg_{cat}⁻¹·h⁻¹ (after 20 hours of slow-activation) with a selectivity (r_{d}/rₕ=rate ratio of dehydrogenation products over hydrogenolysis products) of 10. In embodiments, the catalyst composition is re-usable up-to at least 10 cycles at 873 K demonstrating high-efficiency. The catalyst composition can be used in multiple catalytic cycles and regeneration steps without any noticeable loss in its alkane (or alkene) dehydrogenation rate demonstrating excellent robustness.

According to various embodiments, the Co²⁺ cations provide the active sites of the catalyst compositions. Optimally, the surface density of isolated Co²⁺ cations is high on the catalyst surface to increase the dehydrogenation rate (per mass). During the Co²⁺ grafting procedure, when no vicinal hydroxyl groups are present on the support, CoOₓ crystallites start to form. The formation of such crystallites inhibits the dehydrogenation rate of the catalyst composition with increasing loading of Co²⁺ on the support because the Co-species that do not belong to the surface, do not contribute to the dehydrogenation rate. Controlling the surface density of isolated Co²⁺ species helps optimize performance of the catalyst composition.

Without being bound by any particular theory, it is believed that the presence of cobalt (III) (Co⁻³) cations leads to the formation of Co⁰ during dehydrogenation reaction conditions (e.g., both H₂ and light alkanes are reducing agents). Additionally, the formation of deactivating carbonaceous deposits during the dehydrogenation reaction causes deactivation of the catalyst.

According to embodiments, disclosed herein are methods of preparing a catalyst composition where such methods increase and optimize the isolated Co²⁺ surface density with increasing Co loading on a support, while minimizing vicinal hydroxyl groups (required for Co grafting) on the support. Increasing the surface density of isolated Co²⁺ on the support, results in an increase in dehydrogenation rate per mass of the catalyst composition. High selectivity and catalyst stability predominantly depend on the minimization of Co³⁺ in the catalyst composition, which can be achieved by forming isolated Co²⁺ on support. Controlling the grafting of isolated Co²⁺ on supports together with minimizing the formation of Co³⁺, minimizes the formation of both Co⁰ and deactivating carbonaceous deposits during a dehydrogenation reaction. The resulting catalyst compositions can have undetected deactivation for at least 40 ks (for Co/SiO₂ catalyst with 0.2-0.4 Co nm⁻²) under dehydrogenation reaction conditions.

### Catalyst Compositions

Catalyst compositions as described herein are useful in dehydrogenation reactions, for example, to dehydrogenate light alkane gases to form alkenes. In embodiments, the catalyst compositions can also be used to dehydrogenate light alkene gases to form alkadienes. According to embodiments, the catalyst compositions can comprise cobalt (II) (Co²⁺) cations, for example, in the form of cobalt (II) oxide. The Co²⁺ cations are the active catalyst material in the dehydrogenation reactions according to various embodiments disclosed herein.

The catalyst compositions may also be free of at least one of chromium and a precious metal. Non-limiting examples of precious metals include platinum (Pt), gold (Au), silver (Ag), copper (Cu), palladium (Pd) and combinations thereof In embodiments, the catalyst composition is free of both chromium and platinum. Catalyst compositions as described herein may present fewer risks to human health and the environment than other known catalysts, e.g., chromium- and platinum-based catalysts, used for dehydrogenation.

In embodiments, the catalyst composition can include at least about 0.1 wt % cobalt, at least about 0.5 wt % cobalt, at least about 1.0 wt % cobalt, at least about 2.0 wt % cobalt, at least about 5.0 wt % cobalt, at least about 7.5 wt % cobalt, at least about 10 wt % cobalt, at least about 15 wt % cobalt, or at least about 20 wt % cobalt. In embodiments, the catalyst composition can include about 0.1 wt % to about 20 wt % cobalt, or about 0.25 wt % to about 16 wt % cobalt, or about 0.5 wt % to about 12 wt % cobalt, or about 1.0 wt % to about 10 wt % cobalt, or about 2.0 wt % to about 8.0 wt % cobalt, or about 2.0 wt % cobalt, or about 5.0 wt % cobalt, or about 7.5 wt % cobalt, or about 10 wt % cobalt, or about 15 wt % cobalt, or about 20 wt % cobalt. The amount of cobalt can be determined by calculating how much of CoO will form on a support after degrading from a given number of impregnated Co(II) acetate. The weight (after mass loss) of the supports can be similarly determined when heat treated under conditions of catalyst preparation. The following equation can be used to determine the % wt. of cobalt in each catalyst: (Wt. of CoO in g)/(Wt. of CoO+support in g)).

Catalyst compositions according to embodiments herein contain a plurality of active sites. In embodiments, the active sites are formed by Co²⁺ cations on the surface of a support. The support may be a nominally inert support. The term nominally inert support means that the support provides little or no measurable catalytic activity. In embodiments, the catalytic activity of the support (e.g., in mol/s) is less than about 5% of the catalytic activity of the Co²⁺ cations (e.g., in mol/s). In embodiments, the catalytic activity of the support (e.g., in mol/s) is less than about 4% of the catalytic activity of the Co²⁺ cations (e.g., in mol/s). In further embodiments, the catalytic activity of the support (e.g., in mol/s) is less than about 3% of the catalytic activity of the Co²⁺ cations (e.g., in mol/s). In yet further embodiments, the catalytic activity of the support (e.g., in mol/s) is less than about 2.5%, or less than about 1%, or less than about 0.5% of the catalytic activity of the Co²⁺ cations (e.g., in mol/s). In further embodiments, the catalytic activity of the support (e.g., in mol/s) is less than 0.1% of the catalytic activity of the support (e.g., in mol/s). In embodiments, the support has no measurable catalytic activity. The catalytic activity of the support and of the catalyst composition, for example, as measured in mol/s, is dependent on the type of material (e.g., homogenous, heterogeneous, etc.) as would be understood by those of ordinary skill in the art. According to embodiments, the support comprises a surface density of about 0.1 Co atoms/nm² to about 20 Co atoms/nm². In embodiments, the support comprises a surface density of about 0.25 Co atoms/nm² to about 18 Co atoms/nm². In further embodiments, the support comprises a surface density of about 0.5 Co atoms/nm² to about 15 Co atoms/nm². In yet further embodiments, the support comprises a surface density of about 1.0 Co atoms/nm² to about 10 Co atoms/nm², or about 2.0 Co atoms/nm² to about 8.0 Co atoms/nm². The Co surface density=(% wt. of CoO×6.023×10²³)/(Surface area×74.9×10¹⁸). When he % wt. of CoO is known, the surface area is measured by N₂ physisorption uptakes (e.g., Praxair, 99.999%)) at its normal boiling point in a Quantasorb unit (Quantasorb 6 Surface Analyzers, Quantachrome Corp.) after degassing the samples for 2 h at 423 K.

According one or more embodiments, the support can be a high surface area powder (e.g., gamma, delta or theta alumina powder) comprised of particles, granules and/or spheres (e.g., alumina microspheres or nanospheres in amorphous or colloidal form), which may be referred to herein as supports. In embodiments, the support comprises at least one of silicon dioxide (SiO₂), aluminum oxide (Al₂O₃), fumed Al₂O₃, aluminum oxyhydroxide (AlOₓ(OH)₃₋₂ₓ) and silicon oxyhydroxide (SiOₓ(OH)₂₋ₓ). In embodiments, the support comprises at least one of gamma-Al₂O₃, delta-Al₂O₃ and theta-Al₂O₃.

In embodiments, alumina supports can be activated or transition aluminas. These activated aluminas can be identified by their ordered structures observable by their X-ray diffraction patterns (e.g., measured using a Siemens D5000 unit at ambient temperature with Cu Kα radiation and a scan rate of 0.033° s⁻¹ to determine the crystalline structure of the catalyst composition), which indicate a mixed phase material containing minimal to no low surface area or crystalline phase alpha alumina. Alpha alumina is identified by a defined crystalline phase by x-ray diffraction. The higher surface area activated aluminas are often defined as a gamma alumina phase, but the phase transitions can be a continuum of varying percentages of multiple mixed phases such as, but not limited to, delta and theta phases based on the chosen calcination temperature to achieve the desired support surface area.

According to embodiments, the catalyst compositions are comprised of Co²⁺ oxides dispersed onto SiO₂ and/or Al₂O₃ support materials. As will be discussed in more detail below, an aqueous solution of a Co²⁺ precursor (e.g., cobalt acetate; Co(OAc)₂) may be used to graft Co²⁺ cations onto the SiO₂ and/or γ-Al₂O₃ supports. To maximize performance and achieve the intrinsic activity of the surface Co²⁺ sites, it is important to minimize and/or eliminate surface contamination.

In further embodiments, the catalyst compositions comprising Co²⁺ cations can be dispersed on oxy-hydroxide supports. Without being bound by any particular theory, it is believed that the higher surface density of hydroxyl groups (e.g., as compared to corresponding oxide supports) can increase the surface density of isolated Co²⁺ sites with increasing Co loading.

The Brunauer, Emmett and Teller (BET) surface area of the catalyst composition can be measured using nitrogen (N₂) physisorption uptake at its normal boiling point in a surface analyzer (e.g., a Quantasorb® 6 Surface Analyzer by Quantachrome® Corp.). The BET surface area can be measured as set forth in Otroshchenko, et al., Zro₂-Based Unconventional Catalysts for Non-Oxidative Propane Dehydrogenation: Factors Determining Catalytic Activity, J. of Catalysis, 348, 282-290 (2017), which is incorporated herein by reference in its entirety. Another suitable method of measuring the BET surface area is set forth in ASTM D3663-03(2008), which is incorporated by reference herein in its entirety. Catalyst compositions comprising Co²⁺ as described herein can have a BET surface area of about 1 m² g⁻¹ to about 100 m² g⁻¹, or about 10 m² g⁻¹ to about 90 m² g⁻¹, or about 20 m² g⁻¹ to about 80 m² g⁻¹, 30 m² g⁻¹ to about 70 m² g⁻¹, 40 m² g⁻¹ to about 60 m² g⁻¹, or about 40 m² g⁻¹, or about 45 m² g⁻¹, or about 50 m² g⁻¹, or about 75 m² g⁻¹ to about 355 m² g⁻¹, or about 1 m² g⁻¹ to about 400 m² g⁻¹ as measured by a surface analyzer as described above.

According to various embodiments, the catalyst composition comprising Co²⁺ can further include a rare earth metal. In embodiments, the rare earth metal can include at least one lanthanide metal, an oxide thereof and combinations thereof. According to embodiments, the rare earth metal can be at least one of yttrium (Y), erbium (Er), cerium (Ce), dysprosium (Dy), gadolinium (Gd), lanthanum (La), neodymium (Nd), samarium (Sm), ytterbium (Yb), oxides thereof and mixtures thereof. In embodiments, the catalyst composition can include about 0.5 wt % to about 50 wt %, or about 1 wt % to about 40 wt %, or about 2 wt % to about 30 wt %, or about 3 wt % to about 25 wt %, or about 4 wt % to about 20 wt %, or about 5 wt % to about 15 wt %, or about 1 wt % to about 12 wt %, or about 2 wt % to about 10 wt % of the rare earth metal, an oxide thereof or mixtures thereof. In certain embodiments, the catalyst composition can include yttrium, for example, in the form of yttrium oxide. In embodiments, the rare earth metal increases the surface area of catalyst composition. Such catalyst compositions can comprise about 0.1 wt % to about 20.0 wt % cobalt and about 0.05 wt % to about 15 wt % Y₂O₃ and/or an atomic ratio of the rare earth element to cobalt of greater than 0 to about 0.2. The catalyst composition comprising the Co²⁺ together with the rare earth metal can have a BET surface area of about 40 m² g⁻¹ to about 110 m² g⁻¹.

According to various embodiments, the catalyst composition comprising Co²⁺ can be treated with a pretreatment gas. Pretreating the catalyst composition can increase the number of active sites on the catalyst, which can result in higher catalytic activity during the dehydrogenation reaction (e.g., at the beginning of the reaction). In embodiments, a pretreated catalyst composition containing Co²⁺ comprises more active sites than a catalyst composition containing Co²⁺ that has not been pretreated.

In embodiments, the pretreated catalyst composition can be formed by contacting the catalyst composition with a pretreatment gas under certain conditions. The pretreatment gas can include a reducing agent comprising at least one of H₂, carbon monoxide (CO), ammonia and methane (CH₄). In embodiments, the pretreatment gas can further include an inert gas comprising at least one of nitrogen (N₂), helium (He) and Argon (Ar). In embodiments, the pretreatment gas can comprise the reducing agent at a concentration of about 1 mol % to about 10 mol %, or about 2 mol %, or about 4 mol %, or about 6 mol %, or about 8 mol %, or about 10 mol %. In certain embodiments, the pretreatment gas is H₂ and can include about 1 mol % to about 10 mol % H₂, or about 2 mol % H₂, or about 4 mol % H₂, or about 6 mol % H₂, or about 8 mol % H₂, or about 10 mol % H₂.

During the pretreatment, the pretreatment gas and/or the catalyst composition can be at a temperature of at least about 500 K, or at least about 600 K, or at least about 700 K, or at least about 850 K, or at least about 860 K, or at least about 870 K, or at least about 873 K, or at least about 880 K, or at about 870 K, or at about 871 K, or at about 872 K, or at about 873 K, or at about 874 K, or at about 875 K. The pretreatment gas can be in contact with the catalyst composition for about 0.1 h to about 24 h, or about 0.5 h to about 24 h, or about 1.0 h to about 24 h, or about 2 h to about 22 h, or about 3 h to about 20 h, or about 5 h to about 15 h, or about 8 h to about 12 h, or about 0.1 h, or about 0.5 h, or about 1 h, or about 2 h, or about 3 h, or about 4, hour or about 5 h.

According to various embodiments, catalyst compositions as disclosed herein can be in the form of a plurality of units. The plurality of units can include, but are not limited to, particles, powder, extrudates, tablets, pellets, agglomerates, granules and combinations thereof. The plurality of units can have any suitable shape known to those of ordinary skill in the art. Non-limiting examples of shapes include round, spherical, spheres, ellipsoidal, ellipses cylinders, hollow cylinders, four-hole cylinders, wagon wheels, regular granules, irregular granules, multilobes, trilobes, quadrilobes, rings, monoliths and combinations thereof. In embodiments, the shape of the plurality of units may contributed to the performance of the catalyst composition, for example, by increasing the surface area providing the catalytic activity.

The plurality of units can be formed by any suitable method known to those of ordinary skill in the art. Non-limiting examples of methods for shaping and forming a plurality of units (e.g., from a mixture of catalyst materials) include, extrusion, spray drying, pelletization, agglomeration, oil drop, and combinations thereof In one embodiment, the plurality of units can be formed by pressing a powder into wafers (e.g., at about 690 bar, for about 0.05 h), crushing the wafers and then sieving the resulting aggregates to retain a mean aggregate size of about 100 μm to about 250 or about 1.5 mm to about 5 mm, or about 80 mesh to about 140 mesh.

In certain embodiments, the plurality of units can have a size of less than about 1,000 μm, or less than about 750 μm, or less than about 500 μm, or less than about 300 μm, or less than about 250 μm, or less than about 225 μm, or less than about 200 μm, or less than about 190 μm, or less than about 180 μm, or less than about 150 μm, or less than about 100 μm, or less than about 10 μm as measured by any suitable method known to those of ordinary skill in the art. In embodiments, the plurality of units can have a size of about 170 μm to about 250 μm, or about 80 mesh to about 140 mesh. In further embodiments, the plurality of units have a mean size of about 1.5 mm to about 15.0 mm, or about 1.5 mm to about 12 mm, or about 1.5 mm to about 10 mm, or about 1.5 mm to about 8.0 mm, or about 1.5 mm to about 5.0 mm. Particle size can be measured using any suitable method known to those of ordinary skill in the art. For example, particle size can be measured using ASTM D4438-85(2007) and ASTM D4464-10, both of which are incorporated herein by reference in their entirety.

According to embodiments, catalyst compositions as described herein may be comprised in a kit. The kit can include the catalyst composition as described above and instructions for pretreating the catalyst composition. The instructions can comprise the following elements: 1) place the catalyst composition in a chamber and/or reactor; 2) heat the pretreatment gas, the chamber, the reactor and/or the catalyst composition to a temperature of at least about 850 K, or at least about 860 K, or at least about 870 K, or at least about 873 K, or at least about 880 K, or at about 870 K, or at about 871 K, or at about 872 K, or at about 873 K, or at about 874 K, or at about 875 K; 3) introduce the pretreatment gas into the chamber and/or reactor and contact the catalyst composition with the pretreatment gas for about 0.1 h to about 24 h, or about 0.5 h to about 22 h, or about 1.0 h to about 20 h, or about 2.5 h to about 15 h, or about 5 h to about 12 h, or about 0.1 h, or about 0.2 h, or about 0.5 h, about 1 h, or about 2 h, or about 3 h, or about 4, hour or about 5 h.

According to embodiments, the kit may include the catalyst composition together with instructions for using the catalyst composition in a light alkane (or light alkene) dehydrogenation process. The catalyst composition can be pretreated or may not be pretreated in accordance with embodiments herein. If not pretreated, the kit can further include instructions for pretreating the catalyst composition as described above. The instructions for using the catalyst composition can comprise the following elements: 1) place the catalyst composition in a reactor; 2) introduce the light alkane gas (and/or light alkene gas) together with H₂ into the reactor; and 3) contact the light alkane gas (and/or light alkene gas) and the H₂ with the catalyst composition. Optionally, the instructions may further include 4) recover the dehydrogenated (i.e., alkene or alkadiene) gas.

The kits discussed above can include suitable details and instructions for using the catalyst composition safely and productively. Non-limiting examples of such details and instructions can include how to load the catalyst composition into the reactor, how to pre-treat the catalyst composition, if necessary, before starting the reaction, the starting temperatures and gas composition for bringing the catalyst composition on-stream, the regeneration procedures, how to unload the catalyst composition from the reactor and combinations thereof.

### Methods of Preparing the Catalyst Compositions

According to various embodiments, disclosed herein are methods of preparing catalyst compositions as described above. A catalyst composition comprising Co²⁺ can be prepared by loading cobalt (II) (Co²⁺) cations onto a support. In embodiments, loading the Co²⁺ onto the support can be by at least one of grafting, doping, co-precipitating and impregnating the Co²⁺ cations onto (or within) the support. In embodiments, the loading includes contacting the support with an aqueous solution of at least one of a cobalt (II) carboxylate, a cobalt (II) glycolate and a cobalt (II) citrate. In embodiments, the cobalt (II) carboxylate comprises cobalt (II) acetate tetrahydrate. In embodiments, the aqueous solution is free of a nitrate, CoSO₄ and CoCl₂. Without being bound by any particular theory, it is believed that using a nitrate solution may lead to the formation of Co₃O₄ (of 33.7 nm diameter), possibly because of poor grafting of Co²⁺ on supports, which may result in faster catalytic deactivation (with a deactivation constant (kd) of 2.5 h-1) during a dehydrogenation reaction. It is further believed that CoSO₄ and CoCl₂ may poison the Co-based sites by their respective anions.

The aqueous solution can be at a concentration of about 10 wt % up to the solubility of the at least one of cobalt (II) carboxylate, cobalt (II) glycolate and cobalt (II) citrate in the solution. In embodiments, the aqueous solution is at a concentration of about 10 wt % to about 100 wt %, about 15 wt % to about 90 wt %, about 20 wt % to about 80 wt %, about 25 wt % to about 75 wt %, about 30 wt % to about 60 wt %, or about 40 wt % to about 50 wt % of the at least one of cobalt (II) carboxylate, cobalt (II) glycolate and cobalt (II) citrate by weight of the solution.

In embodiments, the aqueous solution can be at a temperature of about 100 K to about 500 K, or about 150 K to about 450 K, or about 100 K to about 400 K, or about 288 K to about 353 K. The contacting can occur for about 1 h to about 36 h, or about 5 h to about 32 h, or about 10 h to about 24 h, or about 12 h to about 20 h, or about 18 h, or about 19 h, or about 20 h, or about 21 h, or about 22 h.

According to various embodiments, the method further includes, for example, after contacting the support with the aqueous solution, drying the support in ambient air. The drying can be at a temperature of about 350 K to about 450 K, or about 360 K to about 440 K, or about 370 K to about 430 K, or about 380 K to about 420 K, or about 380 K, to about 410 K, or about 390 K to about 400 K, or about 391 K, or about 392 K, or about 393 K, or about 394 K, or about 395 K. In embodiments, the drying can be for about 1 h to about 24 h, or about 2 h to about 22 h, or about 3 h to about 20 h, or about 4 h to about 18 h, or about 5 h to about 16 h, or about 6 h to about 15 h, or about 8 h to about 12 h, or about 9 h to about 10 h.

Following the contacting and/or drying, the support can be heat-treated. The heat-treating can include flowing dry air over the support at a rate of about 1 cm³/s to about 3 cm²/s, about 1.5 cm³/s to about 2.5 cm³/s, or about 2.0 cm³/sec. The heat treating can be at a temperature of about 300 K to about 500 K, or about 325 K to about 450 K, or about 350 K to about 400 K, or about 380 K, or about 381 K, or about 382 K, or about 383 K, or about 384 K, or about 385 K, for a period of about 6 h to about 24 h, or about 8 h to about 22 h, or about 10 h to about 20 h, or about 12 h to about 18 h.

In embodiments, the resulting heat-treated support having the Co²⁺ thereon, can be calcined according to any suitable method known to those of ordinary skill in the art. For example, the heat-treated support can be calcined in flowing dry air (e.g., zero grade) at a flow rate of about 0.5 cm³ s⁻¹ to about 2.00 cm³ s⁻¹, or about 1.67 cm³ s⁻¹ and a temperature of about 700 K to about 1,000 K, or about 750 K to about 950 K, or about 800 K to about 900 K, or about 870 K, or about 871 K, or about 872 K, or about 873 K, or about 874 K, or about 875 K, or about 923 K, for about 1 h to about 6 h, or about 3 h.

In embodiments, a catalyst composition comprising the Co²⁺ cations and a rare earth metal can be prepared according to any suitable method known to those of ordinary skill in the art. For example, a support (e.g., comprising alumina or formed of alumina) can be impregnated with Co²⁺ cations and La(NO₃)₃. Optionally, the impregnated support can be calcined at 600° C. for about 2 hours.

According to embodiments, methods of preparing the catalyst composition can further include pretreating the catalyst composition in a pretreatment gas as discussed above. For example, the catalyst composition can be subjected to a reductive pretreatment, for example, with a pretreatment gas comprising at least one of H₂, carbon monoxide (CO), light alkanes, propane (C₃H₈), alkenes, propene (C₃H₆) and H₂ species present as reactant and products of the dehydrogenation reaction. In embodiments, the catalyst composition can be pretreated with H₂ at a temperature of about 800 K to about 1,000 K, or about 850 K to about 900 K, or about 873 K. The pretreated catalyst composition can include more surface-active sites (e.g., Zr_{cus} surface-active sites) than a catalyst composition that has not been pretreated.

### Methods of Using the Catalyst Compositions

Further described are methods of using catalyst compositions comprising Co²⁺ according to embodiments. In embodiments, the catalyst compositions can be used in the dehydrogenation of hydrocarbons, for example, light alkane gas to form alkenes. In embodiments, the methods can also be used in the dehydrogenation of light alkene gas to form alkadienes. The Co²⁺ present in the catalyst compositions is the active catalyst material in the dehydrogenation reactions as disclosed herein.

In embodiments, when preparing to dehydrogenate a light alkane gas, the catalyst composition can be placed within a reactor (e.g., at a weight hourly space velocity of about 5.5 h⁻¹ to about 0.05 h⁻¹, or about 5.4 h⁻¹ to about 0.054 h⁻¹, or about 2.7 h⁻¹ to about 1.8 h⁻¹, or about 5.0 h⁻¹ to about 0.1 h⁻¹) and held at an about constant temperature using a furnace and a temperature controller (e.g., a Watlow Series 96). The reactor can be any suitable reactor known to those of ordinary skill in the art. Non-limiting examples include a U-shape quartz reactor (e.g., with an inner diameter of about 11.0 mm), a packed tubular reactor, a catofin-type reactor, a fluidized bed reactor, a fixed bed reactor and a moving bed reactor. The furnace may be any suitable furnace known to those of ordinary skill in the art. Non-limiting examples include a single zone furnace (e.g., by National Element Inc., Model No. BA-120), a batchwise furnace or a quartz tube furnace.

Prior to dehydrogenation, the catalyst composition can be treated in a flowing oxygen gas (O₂) and helium (He) mixture at a molar ratio of O₂ of about 20:1 to about 30:1, or about 22:1 to about 28:1, or about 24:1 and heating the flowing gas mixture to a temperature of about 800 K to about 1,000 K, or about 850 K to about 900 K, or about 873 K at a rate of about 0.1 K s⁻¹ to about 1 K s⁻¹, or about 0.167 K s⁻¹. Treating the catalyst composition with the flowing O₂ and He gas mixture can be for a period of about 0.5 h to about 8 h, or about 1 h to about 4 h, or about 1 h, or about 2 h. Subsequently, the reactor can be purged with flowing inert gas (as defined above), steam, or by vacuum (e.g., 2 cm³ g⁻¹ s⁻¹, ultra-high purity) to remove residual O₂ within the reactor.

The light alkane gas (and/or light alkene gas) can be introduced to the reactor in the presence of the catalyst composition. According to embodiments, the light alkane gas (and/or light alkene gas) can comprise any one of a C₂ to C₅ straight or branched alkane and mixtures thereof In embodiments, the light alkane gas (and/or light alkene gas) can comprise at least one of ethane, propane, n-butane, isobutane, pentane and mixtures thereof. In embodiments, a portion of the effluent from the reactor can be recycled to the gas inlet and combined with fresh feed gas. The effluent can comprise alkenes, for example, at least one of ethene, pentene, butene, isobutene and pentene, and unreacted light alkanes comprising at least one of ethane, propane, n-butane, isobutane and pentane. In embodiments, the light alkane gas (and/or light alkene gas) can be mixed with an inert gas (e.g., steam, He, N₂, Ar) at a molar ratio of about 1:2 to about 2:1, or about 1:1. In embodiments, for example, during a Catofin process, a vacuum pump can be used to lower the pressure of the reactants while maintaining the total pressure above, for example, 1 bar, to allow convective flow when the exit pressure is atmospheric.

Methods of dehydrogenating light alkane gas (and/or light alkene gas) can include co-feeding H₂ with the light alkane gas (and/or light alkene gas) in the presence of the catalyst composition. The catalyst composition can comprise Co²⁺ according to various embodiments described herein. Adding or co-feeding the H₂ with the light alkane gas (and/or light alkene gas) can include introducing the H₂ at a pressure of about 1 kPa to about 100 kPa, or about 5 kPa to about 75 kPa, or about 10 kPa to about 50 kPa, or about 30 kPa to about 50 kPa while dehydrogenating the light alkane gas. In embodiments, the H₂ can be added to the light alkane gas (and/or light alkene gas) at a molar ratio of H₂ to light alkane gas (and/or light alkene gas) of about 1:100 to about 1:1. In embodiments, the H₂ and/or reactor can be at a temperature of about 500 K to about 1000 K, or about 550 K to about 950 K, or about 600 K to about 900 K, or about 700 K to about 900 K.

According to embodiments, the method of dehydrogenating the light alkane gas (and/or light alkene gas) in the presence of the catalyst composition as described herein can provide a dehydrogenation rate per mass of the catalyst composition of about 0.5 mol to about 10.0 mol kg⁻¹ h⁻¹, or about 0.6 mol kg⁻¹ h⁻¹ to about 8.3 mol kg⁻¹ h⁻¹. The light alkane gas (and/or light alkene gas) dehydrogenation rate and cracking rate can be determined by analyzing the effluent stream from the reactor using gas chromatography (e.g., by an Agilent® 1540A gas chromatograph) with flame ionization detection (FID) (e.g., a GC fitted with a GS-GASPRO column) after chromatographic separation. In embodiments, the light alkane gas (and/or light alkene gas) dehydrogenation rate and the cracking rate can be normalized by the mass of the catalyst composition (e.g., in mol kg⁻¹ h⁻¹). In embodiments, the light alkane gas (and/or light alkene gas) dehydrogenation rate and cracking rate can be determined at a temperature of 873 K and 823 K. The temperature can be measured using any suitable method known to those of ordinary skill in the art. In embodiments, the temperature can be measured with a thermocouple (e.g., a K-type thermocouple by Omega®) and the reactor temperature can be determined from a thermocouple placed in contact with an outer tube surface (e.g., made of metal, quartz, etc.) at the catalyst bed midpoint.

In embodiments, when used in a dehydrogenation reaction as described above, catalyst compositions as disclosed herein can provide improved stability over other known catalyst compositions for the dehydrogenation of light alkanes (and/or light alkenes). The half-life of the catalyst composition in the dehydrogenation reaction can be measured using any suitable method known to those of ordinary skill in the art. In embodiments, the term half-life of the catalyst composition can refer to the number of days or hours after which the catalyst device has a dehydrogenation rate (DR) that is 50% lower than an initial or maximum dehydrogenation rate (DR) value produced by the catalyst composition at the start (or soon after the start upon stabilization) of the catalyst composition's operation (e.g., the half-life of the catalyst composition can be based on the dehydrogenation reaction rate as a function of time). In embodiments, the half-life of the catalyst composition is related to the weight hourly space velocity (WHSV), which is the hourly mass feed flow rate per catalyst mass (h⁻¹) in the reactor. In embodiments, the catalyst composition can have a half-life of about 1 h to about 50 h, or about 6 h to about 46 h when the WHSV is about 5.4 h⁻¹ to about 0.054 h⁻¹.

The half-life of the catalyst composition can also be evaluated when aging the composition under different conditions. The DR can be calculated based on Formula I:

DR=60·(kₑ−kₙ)·V Formula I

wherein kₑ represents the total decay constant, kₙ represents natural decay constant, V represents the chamber volume in m³, and (kₑ−kₙ) is calculated based on Formula II:

(kₑ−kₙ)·t=−ln(Cₜ/C₀) Formula II

wherein t represents the total testing time, Cₜ represents the concentration at time t in mg/m³, and C₀ represents the concentration at time t=0 in mg/m³.

To determine the half-life of the catalyst composition, testing can begin by obtaining the DR value produced by the catalyst composition at the start of the catalyst composition's operation or soon after the start of the catalyst composition's operation once the DR has stabilized (t=0), also known as DR₀. Subsequently, the catalyst composition can be optionally subjected to an Acceleration Test. An Acceleration Test refers to an extreme condition that may impact or deteriorate the efficacy of the catalyst composition more rapidly, such as no H₂ gas co-feed or pre-treatment, co-feeding with O₂, introducing a pollutant or continuous generation of pollutants. The optional Acceleration Test results can allow the estimation of the catalyst composition's life span under real-life conditions. Following the optional Acceleration Test, the catalyst composition is then aged under real-life conditions.

After the catalyst composition is aged for eight hours under real-life conditions, another sample is taken to obtain the DR value at t=n, also known as DRₙ. If the DRₙ value is greater than 50 percent of the DR₀ value, then the catalyst composition is considered as still operable and the testing continues by repeating the optional acceleration test, aging the catalyst device under real-life conditions, and measuring the DRₙ value after each subsequent cycle. Once the DRₙ value is lower or equal to 50 percent of the DR₀ value, the life span of the catalyst device is deemed to have ended and the overall alkene mass (AM) generated by the catalyst composition is calculated.

The above-described methods of using the catalyst compositions to dehydrogenate light alkane gas, can also be used to dehydrogenate a light alkene gas to form alkadienes. A light alkene gas can include C₂-C₅ branched or straight alkenes. In embodiments, two reactors can be configured in series, the first for dehydrogenating light alkane gas and the second for dehydrogenating the light alkene gas.

Catalytic dehydrogenation reactions can be classified into two categories, based on the presence of dioxygen in the reaction environment; they are denoted as oxidative and non-oxidative dehydrogenation processes. Oxidative dehydrogenation reactions occur in the presence of oxygen-containing gases and form combustion side products along with alkenes; non-oxidative dehydrogenations occur in the absence of oxygen-containing gases and form hydrogenolysis side products and carbonaceous residues that tend to block active sites.

According to embodiments, the method of using the catalyst composition comprising Co²⁺ as described herein is a process for non-oxidative dehydrogenation of light alkane (or light alkenes) in the presence of hydrogen (H₂) to produce corresponding alkenes using SiO₂ and Al₂O₃ supported Co-based catalyst compositions. The catalyst compositions can be synthesized in a single step as described above to provide cobalt in its intermediate oxidation state (Co²⁺), which is active for the non-oxidative dehydrogenation processes. In embodiments, any gas containing oxygen (O₂) was avoided during propane dehydrogenation in order to eliminate the possibility of hydrocarbon oxidation to CO or CO₂. In embodiments, no catalyst deactivation was detected for at least 40 ks (when performed on Co/SiO₂ catalyst with 0.2-0.4 Co nm⁻²). The dehydrogenation rate (per mass) can increase with time (for a prolonged period of time) showing no indication of decrease. This is applicable for materials with low cobalt content (up to around 0.5 Co nm⁻²) and may not be observed for materials with higher cobalt loading.

In embodiments, the dehydrogenation of straight or branched light alkanes with catalyst compositions as described herein can provide higher dehydrogenation rates than other known Co-based catalysts, with higher selectivity and stability, and with the robustness required for occasional oxidative regenerations. Such catalyst compositions can be formed, for example, using SiO₂ and γ-Al₂O₃ supported Co(II)-based catalysts, in the presence of hydrogen (H₂) at the high temperatures required by the thermodynamics of these very exothermic dehydrogenation reactions.

### EXAMPLES

### Example 1

### Preparing Catalyst Compositions Comprising Co²⁺

CoOₓ/SiO₂ catalysts were prepared by impregnating SiO₂ (Sigma-Aldrich, high-purity grade, 293 m² g⁻¹) with an aqueous solution of cobalt (II) acetate tetrahydrate (Sigma-Aldrich, 99.99%). Samples were then dried at 393 K in ambient air for 9 h and treated in flowing dry air (Praxair, zero grade, 1.67 cm³ s⁻¹) at 923 K for 3 h.

CoOₓ/Al₂O₃ catalysts were prepared by impregnating fumed γ-Al₂O₃ (Catalox SBA-90 Alumina, 110 m² g⁻¹) with an aqueous solution of cobalt (II) acetate tetrahydrate (Sigma-Aldrich, 99.99%). Samples were dried at 393 K in ambient air for 9 h and treated in flowing dry air (Praxair, zero grade, 1.67 cm³ s⁻¹) at 923 K for 3 h.

AlOₓ(OH)₃₋₂ₓ supports (121 m² g⁻¹) were prepared by the hydrolysis of an aqueous solution of 0.5 M Al(NO₃)₃ (Aldrich Chemicals, >98%) pH 10, controlled by 14 N NH₄OH (Aldrich Chemicals, >98%). The precipitate was washed thoroughly by an aqueous solution of NH₄OH (pH 10) until residual Cl⁻ ions were removed, tested by adding the filtrate to a 3 M AgNO₃ solutions which forms white AgCl precipitate in the presence of Cl⁻ ions at a concentration at and above 10 ppm). The AlOₓ(OH)₃₋₂ₓ precipitate was then dried at 393 K for 12 h prior to further treatments to preparing the catalyst.

CoOₓ/AlOₓ(OH)₃₋₂ₓ catalysts were prepared by impregnating AlOₓ(OH)₃₋₂ₓ (121 m² g⁻¹) with an aqueous solution of cobalt (II) acetate tetrahydrate (Sigma-Aldrich, 99.99%). Samples were dried at 393 K in ambient air for 9 h and treated in flowing dry air (Praxair, zero grade, 1.67 cm³ s⁻¹) at 923 K for 3 h. All samples were additionally treated before catalytic reactions using the procedures described below.

All catalysts were treated in flowing dry air (Praxair, zero grade, 1.67 cm³ s⁻¹) at 923 K for 3 h before the BET surface area and powder X-ray diffractogram measurements. The BET surface area of the catalysts was measured using N₂ physisorption uptakes (Praxair, 99.999%)) at its normal boiling point in a Quantasorb unit (Quantasorb 6 Surface Analyzers, Quantachrome Corp.) after degassing the samples for 2 h at 423 K.

Powder X-Ray diffractograms (PXRD) were measured with a Siemens D5000 diffractometer at ambient temperature using Cu Kα radiation with a scan rate of 2° min⁻¹. The Co surface density, the number of Co atoms per nm² of surface area (Co atoms nm⁻²) of the catalyst was obtained by the equation:

Co surface density=(% wt. of CoO×6.023×10²³)/(Surface area×74.9×10¹⁸),

where, the unit of the surface area is m²g⁻¹. The BET surface areas of all Al₂O₃ and SiO₂ supported materials (treated in flowing dry air at 923 K for 3 h) have been summarized in Table 1 and Table 2, respectively.

TABLE 1
BET Surface Areas and CoOx
Surface Density of Co/Al₂O₃ Catalysts^{a}

| | | | Surface | Surface | CoOx |
|---|---|---|---|---|---|
| | | | area | area | surface |
| | Deno- | % wt., | (m² g⁻¹ | (m² g⁻¹ | density |
| Catalysts | tation | CoOx | cat) | Al₂O₃) | (Co nm⁻²) |
| 0.3% CoOₓ/Al₂O₃ | 0.3CoAl | 0.3% | 105 | 105 | 0.2 |
| 1% CoOₓ/Al₂O₃ | 1CoAl | 1% | 95 | 96 | 0.8 |
| 2% CoOₓ/Al₂O₃ | 2CoAl | 2% | 95 | 97 | 1.7 |
| 4% CoOₓ/A1₂O3 | 4CoAl | 4% | 87 | 91 | 3.7 |
| 16% CoOₓ/Al₂O₃ | 16CoAl | 16% | 78 | 93 | 16 |

^{a}All catalysts were treated in flowing dry air (Praxair, zero grade, 1.67 cm³ s⁻¹) at 923K for 3 h.

TABLE 2
BET Surface Areas and CoOx Surface
Density of Co/SiO₂ Catalysts^{a}

| | | | Surface | Surface | CoOx |
|---|---|---|---|---|---|
| | | | area | area | surface |
| | Deno- | % wt., | (m² g⁻¹ | (m² g⁻¹ | density |
| Catalysts | tation | CoOₓ | cat) | SiO₂) | (Co nm⁻²) |
| 0.4% CoOₓ/SiO₂ | 0.4CoSi | 0.4% | 266 | 267 | 0.1 |
| 0.8% CoOₓ/SiO₂ | 0.8CoSi | 0.8% | 259 | 261 | 0.2 |
| 1.5% CoOx/SiO₂ | 1.5CoSi | 1.5% | 251 | 254 | 0.4 |
| 2.5% CoOₓ/SiO₂ | 2.5CoSi | 2.5% | 241 | 247 | 0.8 |
| 4.5% CoOₓ/SiO₂ | 4.5CoSi | 4.5% | 235 | 246 | 1.5 |
| 8.2% CoOₓ/SiO₂ | 8.2CoSi | 8.2% | 219 | 238 | 3.1 |
| 16% CoOₓ/SiO₂ | 16CoSi | 16% | 202 | 240 | 6.2 |

^{a}All catalysts were treated in flowing dry air (Praxair, zero grade, 1.67 cm³ s⁻¹) at 923K for 3 h.

FIG. 1 shows the UV-visible spectra of the 0.2 CoAl, 0.8 CoAl, 3.7 CoAl and 16 CoAl catalyst compositions. Diffuse reflectance UV-visible spectra were collected with a Cary 4 spectrophotometer (Varian Corp.) equipped with a Harrick Scientific diffuse reflectance attachment (DRP-XXX) and a reaction chamber (DRA-2CR). Samples were treated in 20% O₂-He (Praxair, 99.999%, 1.33 cm³ s⁻¹) at 523 K for 0.5 h before measurements. The Kubelka-Munk function (F(R∞)) was used to convert reflectance data into pseudo-absorbance using MgO as a reflective standard. Calculation of absorption-edge energies (for CoOₓ/Al₂O₃) from the x-intercept of a linear regression of [(F(R_{∞}))hν]^{1/2} versus hν were not performed because the broad adsorption bands arising from O to Co³⁺ (ligand to metal) charge transfer masked the LMCT band arising from O to Co²⁺ charge transfer. The presence of three distinct adsorption feature between 1.8-2.5 eV is indicative of the presence of Co²⁺ in the synthesized materials.

### Example 2

### Dehydrogenation of Propane Using Prepared Catalyst Compositions

The rates and selectivities of non-oxidative dehydrogenation of propane on SiO₂ and Al₂O₃ supported CoOₓ catalysts (0.1-0.2 g), sieved to 180-250 μm aggregates, were measured at different temperatures between 673-923 K in a tubular quartz reactor with plug-flow hydrodynamics. The temperature of the reactor was set using an electrical furnace, coupled with a temperature controller (Watlow, series 988), and was measured by a K-type thermocouple (Omega) inserted into the furnace and positioned in the dimple of the reactor wall.

Reactant mixtures contained C₃H₈ (Praxair, 49.3%, balance He) and H₂ (Praxair, 99.999%) and He (Praxair, 99.999%). All gases were metered using mass flow controllers (parker-porter instruments) at flow rates adjusted to get desired C₃H₈ and H₂ pressures (0-60 kPa).

Catalysts were treated in flowing dry air (Praxair, zero grade, 1.67 cm³ s⁻¹) for 0.5 h at 873 K and then in He (Praxair, ultra-high pure, 1.67 cm³ s⁻¹) for 0.5 h at the same temperature to remove residual O₂. Followed by this, Propane (50% C₃H₈ balanced with He, Praxair), Hydrogen (UHP, Praxair), and He (UHP, Praxair) were introduced to the reactor for catalytic measurements.

Reactant and product concentrations were measured by gas chromatography (Agilent 6890) using a methyl siloxane capillary column (HP-1, 50 m×0.32 mm×1.05 μm) connected to a flame ionization detector. Methane (CH₄), ethane (C₂H₆), Ethylene (C₂H₄), propene (C₃H₆), and propane (C₃H₈) were the only products detected.

Dehydrogenation rates of propane (normalized by per mass (kg⁻¹) or atom (Co nm⁻²)) were defined by the rates of propene formation (r_{d}=rₚᵣₒₚₑₙₑ) and the hydrogenolysis rates (rₕ) were determined from the rates of methane, ethane and ethylene formation (rₕ=rₘₑₜₕₐₙₑ/3+2 (rₑₜₕₐₙₑ+r_{ethylene})/3). Reactions using pure SiO₂ and Al₂O₃ showed that the reaction rates were negligible in the absence of CoOₓ species. The gas-phase reactions contributing towards the dehydrogenation and hydrogenolysis reactions, were minimized by decreasing the dead-volume of the reactor using quartz tubes and measured using a catalyst-free reactor; and the dehydrogenation and hydrogenolysis rates (mol h⁻¹) obtained in the presence of the catalyst were subtracted by their corresponding gas-phase values prior to normalizing them by the mass or atoms of the catalyst used.

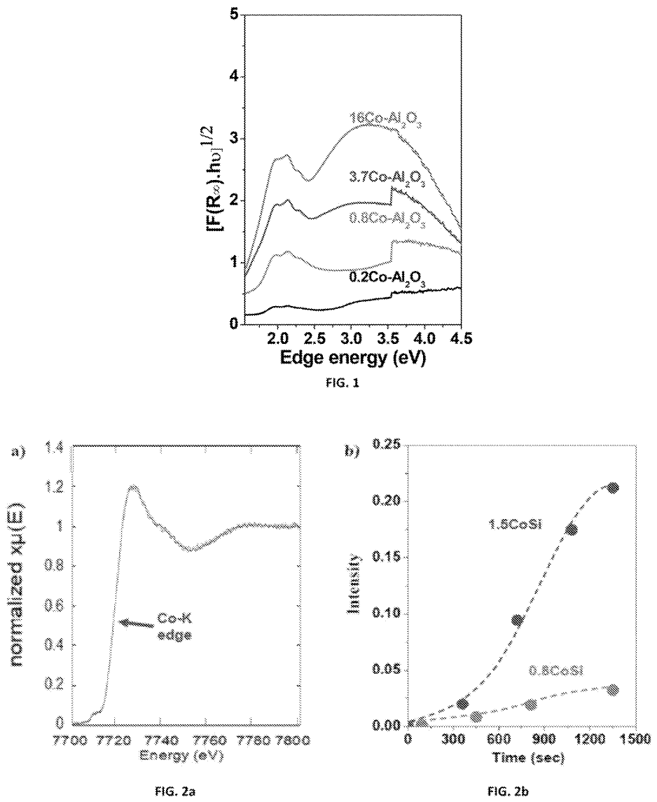

FIG. 2a shows the in-situ X-ray absorption spectra of the 0.8 CoSi catalyst composition and FIG. 2b shows the change of the K-edge intensity of Co as a function of time (sec) in H₂ at 873 K for 0.8 CoSi, and 1.5 CoSi catalysts. In-situ X-ray absorption spectra were collected on the CoSi catalysts in reaction condition, where the Co-K edge intensity was found to decrease, suggesting the reduction of some of the Co-sites, more dramatically with increasing Co loading in the CoSi catalysts (FIG. 2b) when treated in 101.3 kPa H₂ at 873 K. This indicates the presence of more reducible Co' sites besides the irreducible Co²⁺ sites in CoSi catalysts and their (Co³⁺ sites) increase with increasing Co surface density, which is also consistent with the increasing overlap of LMCT bands of CoO (O to Co²⁺) and Co₂O₃ (O to Co³⁺), as observed from UV-Vis spectroscopy.

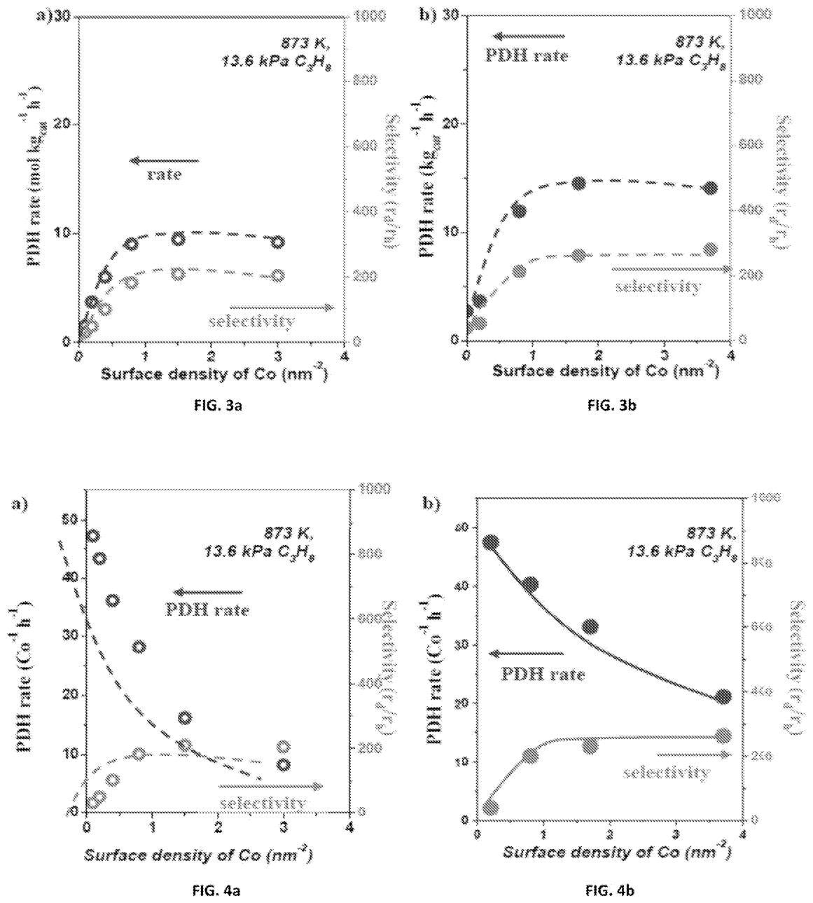

FIG. 3a shows the effect of CoOₓ surface density of the rate (per mass) of a CoSi catalyst composition at reaction conditions: 873 K, 13.6 kPa PC3H8. FIG. 3b shows the effect of CoOₓ surface density of the rate (per mass) of a CoAl catalyst at reaction conditions: 873 K, 13.6 kPa PC₃H₈. Both the CoSi and CoAl catalysts were pre-treated at the following pre-treatment conditions: 873 K, 101.3 kPa PHe, 0.5 h. CoSi and CoAl materials with a wide range of Co surface density were tested under the reaction conditions provided above, and an increasing rate (per mass) of dehydrogenation was obtained with increasing surface density of cobalt (Co) up-to a certain value after which rates (per mass) did not increase further. Such trends indicate that:

The initial increment in the PDH rate (per mass) with increasing CoOₓ surface density is associated with the formation of increasing amounts of isolated Co²⁺ on support. Beyond a certain surface density, no more isolated Co²⁺ forms with increasing Co loading because of the absence of vicinal hydroxyl (—OH) groups on the support, leading to the formation of two-dimensional structures and then three-dimensional clusters of CoOₓ (CoOₓ crystallites), which keeps the active sites beneath the surface layer inaccessible to the reactants, and that in turn, causes the formation of the plateaus as observed in FIG. 2.

FIG. 4a shows the effect of CoOₓ surface density of the rate (per Co) and selectivity of a CoSi catalyst at a reaction condition: 873 K, 13.6 kPa PC₃H₈. FIG. 4b shows the effect of CoOₓ surface density of the rate (per Co) and selectivity of a CoAl catalyst at the same reaction condition: 873 K, 13.6 kPa PC₃H₈. Both the CoSi and CoAl catalysts were pretreated under the following pre-treatment condition: 873 K, 101.3 kPa PHe, 0.5 h. CoSi and CoAl materials with a wide range of Co surface density were tested under the reaction conditions provided above, and a decreasing rate (per Co) of dehydrogenation was obtained which could be explained by the following points: 1) The PDH rate (per Co) decreased rapidly with increasing surface loading of CoOₓ. Increasing surface loading of CoOₓ causes the formation of CoOₓ crystallites, whose growth makes an increasing amount of Co²⁺ inaccessible to the reactants. This caused the PDH rate (per Co) to decrease rapidly with increasing surface loading of CoOₓ, probably because of both the smaller fraction of Co at surfaces and their more facile reduction to Co metal; and 2) the comparison of PDH rate (per Co) at similar loadings of CoOₓ on different supports (SiO₂ and Al₂O₃) yielded similar results, which implies the lack of contribution of corresponding supports towards the PDH reaction.

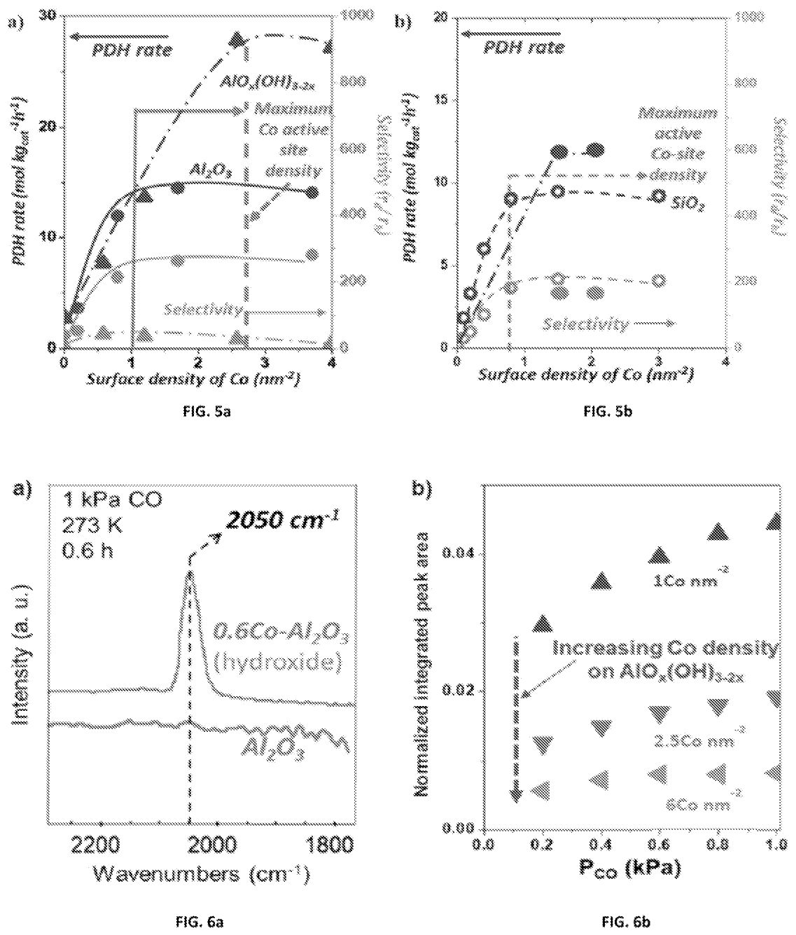

FIGS. 5a and 5b show a comparison of the rate of dehydrogenation of propane (C₃H₈) (per mass) between a) Al₂O₃ and Aluminum oxy-hydroxide supported Co-catalysts, and b) SiO₂ and Silicon oxy-hydroxide supported Co-catalysts, at reaction conditions: 873 K, and 13.6 kPa PC₃H₈. The catalysts were pretreated under the following pre-treatment conditions: 873 K, 101.3 kPa PHe, 0.5 h.

According to FIG. 2b, the PDH rate (per mass) increased with increasing Co loading until reaching a maximum Co²⁺ surface density of about one Co nm⁻², beyond which no further increment in the PDH rate (per mass) was observed because with increasing Co loading the surface density of isolated Co²⁺ sites did not increase any further. This led to the use of aluminum and silicon oxy-hydroxide supports, where the surface density of hydroxyl (—OH) groups are higher than corresponding oxide supports, to impregnating Co²⁺ on it and explore whether increased vicinal hydroxyl groups on the oxy-hydroxide support help to achieve a higher surface density of isolated Co²⁺ sites than that of a corresponding oxide support. This was evaluated by comparing the PDH rates (per mass) (as shown in FIG. 4) of Al₂O₃ and SiO₂ supported Co-catalysts with the corresponding oxy-hydroxide supported Co-catalysts, at reaction conditions (the catalyst compositions were prepared using the method of Example 1).

FIG. 6a shows the infrared spectra of CO adsorbed on aluminum oxy-hydroxide supported CoOₓ catalysts at 268-273 K (1.0 kPa CO, 99.0 kPa He) after treatment in flowing He (0.7 cm³g⁻¹ s⁻¹) at 473 K for 1 h. FIG. 6b shows the integrated CO adsorption peak areas for differently loaded aluminum oxy-hydroxide supported CoOₓ catalysts measured at 268-273 K over the range 0.2-1 kPa CO. The aluminum oxy-hydroxide supported CoOₓ catalysts showed infrared bands at 2050 cm⁻¹ (after subtracting gas-phase CO), whereas the Co²⁺-exchanged LTA samples were reported to have the CO adsorption at 2180 cm⁻¹. See Shee, et al., Light Alkane Dehydrogenation Over Mesoporous Cr₂O₃/Al₂O₃ Catalysts, Appl. Catal. A Gen. 389 (2010) 155-164. The lowering of this CO-adsorption frequency from 2180 cm⁻¹ to 2050 cm⁻¹ is attributed to the presence of —OH on the CO adsorbed Co²⁺. See Saito, et al., Dehydrogenation of Propane Over a Silica-Supported Gallium Oxide Catalyst, Catal. Letters. 89 (2003) 213-217. The Co—CO bands at different CO pressures (in the range of 0.2-1.0 kPa) were integrated and normalized by Al₂O₃ framework peaks and Co surface densities which showed a decreasing integrated peak area (normalized) with increasing Co density, which, in turn, suggests the monomeric CoOₓ species to be the predominant CO adsorption sites.

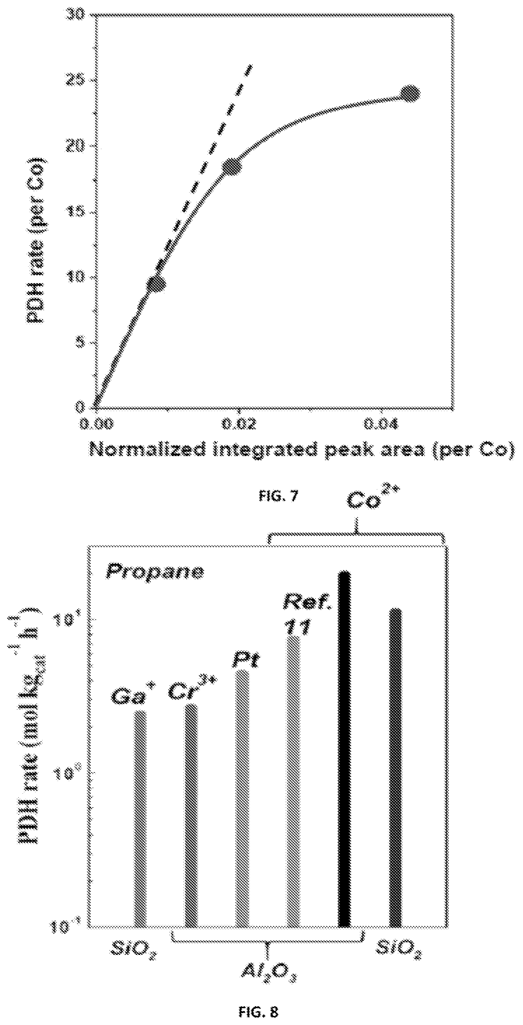

FIG. 7 shows the PDH rate (per Co) as a function of CO-IR area at saturation (per Co) at 268-273 K for aluminum oxy-hydroxide supported CoOₓ catalysts, after treatment in flowing He (0.7 cm³g⁻¹ s⁻¹) at 473 K for 1 h. During the calcination of the alumina oxy-hydroxide supported CoOₓ catalysts, it is presumable that some of the Co-sites will go to the bulk whereas the rest of the Co-sites will stay on the surface. Those surface Co-sites, however, can be considered as equally active for PDH reactions, if the PDH rates (per Co) plotted as a function of the CO-IR peak area at saturation (per Co) falls on a straight line passing through the origin. In reality, when the PDH rates (per Co) were plotted as a function of the CO-IR peak area at saturation (per Co), the trend-line was found to deviate from a straight line (passing through origin) with decreasing Co surface density, indicating the formation of different surface Co-sites with increasing Co surface density.

FIG. 8 shows a comparison of the rate of dehydrogenation of propane among different catalytic systems, details of the catalysts and the reaction conditions are mentioned in Table 3.

TABLE 3
Summary of the Catalytic Data of Different Systems
for Non-Oxide-Dehydrogenation of Propane.

| | Reaction | Feed gas | Rate | Selectivity | K_{d} | |
|---|---|---|---|---|---|---|
| Catalysts | temperature | composition | (molKg⁻¹h⁻¹) | (%) | (h⁻¹) | ref |
| 1CoAl | 873 K | 13.6 kPa | 16 | 93 | n.d. | This |
| | | PC₃H₈. | | | | work |
| 2.5CoSi | 873 K | 13.6 kPa | 11 | 89 | n.d. | This |
| | | PC₃H₈. | | | | work |
| Co-Al- | 873 K | 10 kPa | 5.2 | 81 | 2.5 | [11] |
| mixed metal | | 91 kPa PN₂ | | | | |
| oxide | | | | | | |
| 1% wt. Pt/ | 873 K | 50 kPa | 3.2 | 60 | 0.23 | [16] |
| SiO₂ | | PC₃H₈. | | | | |
| 9% wt. Cr₂O₃/ | 580 K | 5 kPa | 1.7 | 98 | 0.52 | [17] |
| Al₂O₃ | | PC₃H₈. | | | | |
| | | 96 kPa P_{He} | | | | |
| 1.7% wt. Ga₂O₃/ | 823 K | 10 kPa | 1.5 | 86-80 | 0.04 | [18] |
| SiO₂ | | PC₃H₈. | | | | |
| | | 91 kPa P_{Ar} | | | | |

The CoSi and CoAl materials with highest PDH rates (per mass) (that is 1.5 CoSi and 1 CoAl) were compared with the CoAl material reported earlier and other Ga⁺, Cr³⁺, and Pt-based catalysts. Support of each system has been mentioned in FIG. 7. The dehydrogenation reactions in all cases were performed under similar conditions as mentioned above. The PDH rate (per mass) was found to be higher for Co²⁺ based systems as compared to other Ga⁺, Cr³⁺, and Pt-based catalysts. Whereas, the lower PDH rate (per mass) of the mesoporous Co—Al spinel catalyst, (see Hu, et al., A Mesoporous Cobalt Aluminate Spinel Catalyst for Nonoxidative Propane Dehydrogenation, ChemCatChem. 9 (2017) 3330-3337) as compared to the 1.5 CoSi and 1 CoAl catalysts, was probably due to the presence of a smaller number of isolated Co²⁺ species grafted at support surfaces.

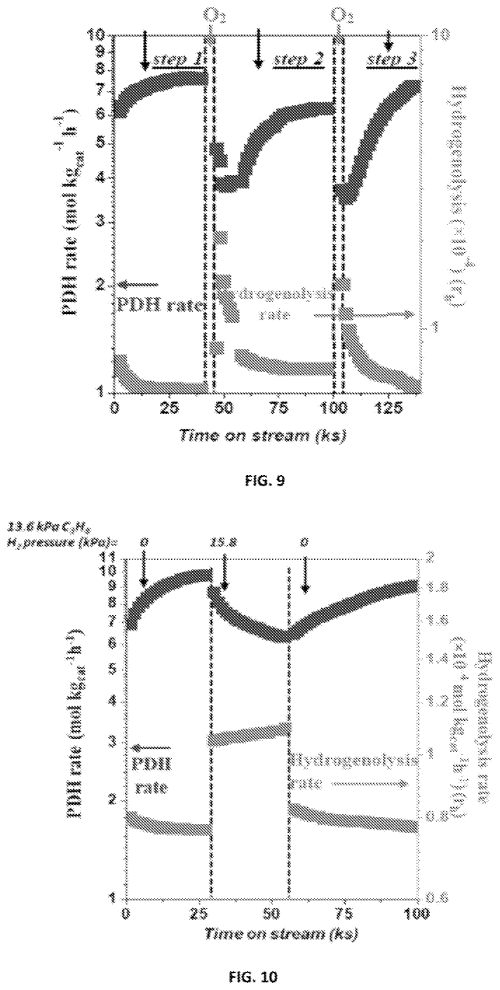

FIG. 9 shows the change and stability of propane dehydrogenation rate (per mass) on 0.8 CoSi at reaction conditions: 873 K, 13.5 kPa PC₃H₈, 0-15.8 kPa PH₂. The catalyst was pretreated under the following pre-treatment condition: 873 K, 101.3 kPa PHe, 0.5 h. The 0.8 CoSi catalyst was run under the reaction conditions provided above to determine its stability in PDH reactions. The findings show a long activation period was obtained, after each catalyst regeneration step (treated in O₂ or dry air) which can be referred to as the formation of active Co²⁺ sites through the reduction of inactive Co³⁺ sites in the reaction conditions (where both H₂ and propane are reducing agents). No catalyst deactivation was observed for the 0.8 CoSi catalyst even after running for a long period of time (for at least 40 ks). The PDH rate (8 mol·Kg⁻¹·h⁻¹) attained for the catalyst in certain reaction conditions, can be re-achieved after regeneration of the catalyst by O₂ treatment, even after performing reactions for more than 50 h in H₂ in between the cycles (FIG. 7, shows the re-attainment of the stable PDH rate (per mass) of step 1 by 0.8 CoSi catalyst in the end of step 3). The similarities of the PDH rates (in same reaction conditions) upon regeneration, validates the reusability of catalyst compositions according to embodiments herein.

FIG. 10 shows the effect of hydrogen (H₂) pressure on the rate of dehydrogenation and hydrogenolysis of propane on a 1.5 CoSi catalyst at reaction conditions: 873 K, 13.6 kPa PC₃H₈, 0-16 kPa PH₂ and pre-treatment conditions: 873 K, 101.3 kPa PHe, 0.5 h. The propane dehydrogenation rates (per mass) changed very slowly with hydrogen pressure. The PDH rates were found to drop with increasing H₂ pressure which could be because of the over reduction of the catalyst. This led to pre-treating the catalyst in 101.3 kPa H₂ for overnight and redo the H₂ pressure dependence study.

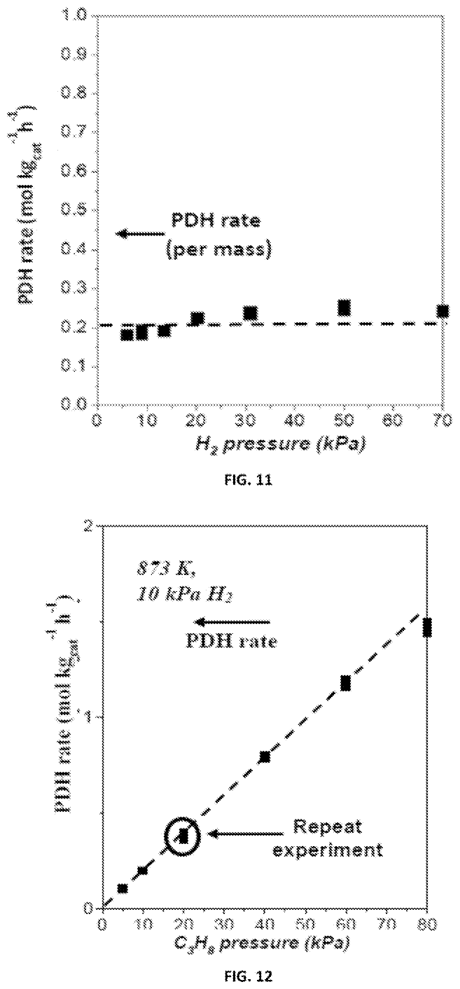

FIG. 11 shows the effect of hydrogen (H₂) pressure on the rate of dehydrogenation of propane on 0.8 CoSi catalyst at reaction conditions: 873 K, 13.6 kPa PC₃H₈, 5-70 kPa PH₂ and pre-treatment conditions: 873 K, 101.3 kPa PH₂, 12 h. The rate of dehydrogenation of propane (per mass) was found to have a zero-order rate dependence on H₂ pressure in the range of 5-70 kPa.

FIG. 12 shows the effect of propane (C₃H₈) pressure at the rate (per mass) of propane dehydrogenation reaction of 1.5 CoSi catalyst at reaction conditions: 873 K, 5-80 kPa PC₃H₈. Pre-treatment condition: 873 K, 101.3 kPa PH₂, 12 h. The rate of dehydrogenation of propane (per mass) was found to have a first (1^{st}) order rate dependence to the propane pressure in the range of 5-80 kPa.

### Elementary Steps and the Rate Equation of PDH CoSi:

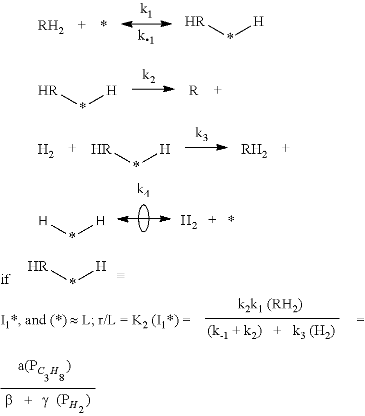

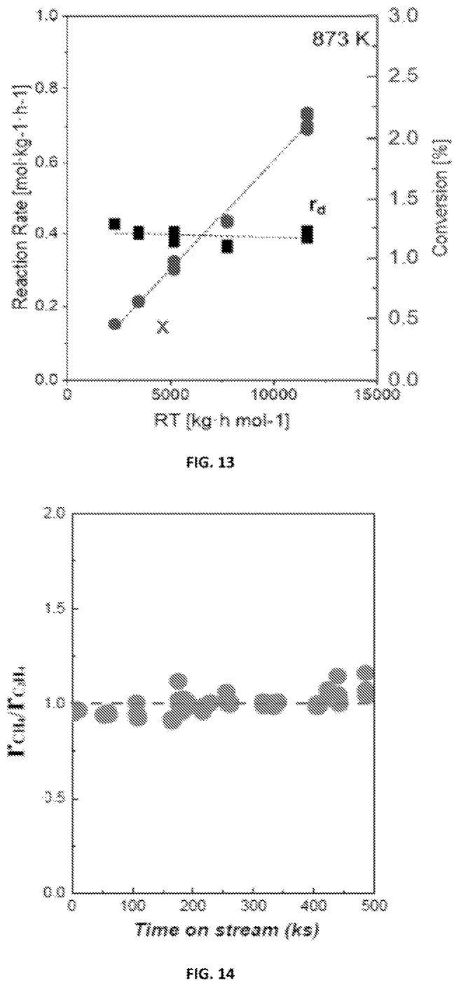

FIG. 13 shows the effect of residence time (RT) on the rate (per mass) of propane dehydrogenation reaction of a 0.8 CoSi catalyst at reaction conditions: 873 K, 15 kPa H₂, and 22.5 kPa PC₃H₈ and a pre-treatment condition: 873 K, 101.3 kPa PH₂, 12 h. The 0.8 CoSi material (of 60-10 mesh size) was tested at varying residence time (RT) in the range of 2500 to 12500 kg·h·mol⁻¹ at 873 K, to observe the effect of propene inhibition to the PDH rate (per mass). The conversion of propane was found to increase with increasing residence time, which in turn, indicates the increasing amount of propene formation, which however was not found to decrease the PDH rate (per mass), suggesting the absence of propene inhibition effect.

### Reaction Network of PDH CoSi:

steps: 1. C₃H₈→C₂H₄+CH₄

2. C₃H₈+2 H₂→3CH₄

3. C₃H₈→C₃H₆

From 1 and 2: rCH₄=r_{c}+3 rₕ=(k_{c}+3 kₕ PH₂²)PC₃H₈

From 1: rC₂H₄=kc PC₃H₈

From 1: rCH₄/rC₂H₄=(k_{c}+3 kₕ PH₂²)/k_{c}=1+(3 kₕ)/k_{c} PH₂²=1+K PH₂²

FIG. 14 shows the contribution of catalytic cracking reaction of propane on a 1.5 CoSi catalyst to form methane and ethylene at reaction conditions: 873 K, 8.3-15 kPa PC₃H₈, 0 kPa PH₂ and pre-treatment conditions: 873 K, 101.3 kPa PHe, 0.5 h. The 1.5 CoSi material (of 60-10 mesh size) was tested at varying propane pressures in the range of 8.3 to 15 kPa at 873 K, to observe the contribution from catalytic cracking in the absence of H₂. According to the reaction network, the predicted rate ratio of methane to ethylene (that is rCH₄ to rC₂H₄) should be 1, if they are forming though the catalytic cracking reactions. The reactions were performed as per the reaction conditions mentioned above and the rate ratio of methane to ethylene (that is rCH₄/rC₂H₄) was found to be around 1 at all conditions for a total run time of 500 ks. This validates the possibility of catalytic cracking reaction in the formation of methane and ethylene.

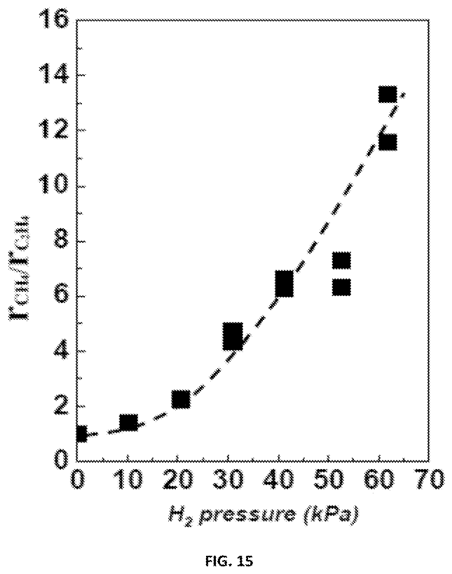

FIG. 15 shows the effect of hydrogen (H₂) pressure on the rate ratio of methane and ethylene on a 1.5 CoSi catalyst at reaction conditions: 873 K, 8.3-15 kPa PC₃H₈, 0-60 kPa PH₂ and a pre-treatment condition: 873 K, 101.3 kPa PHe, 0.5 h.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the scope of the present invention.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should be determined with reference to the appended claims, along with the full scope of equivalents to which such claims are entitled.

1-73. (canceled)

74. A catalyst composition comprising:
a support comprising cobalt (II) (Co²⁺) cations,
wherein the catalyst composition is free of at least one of chromium and a precious metal.

75. The catalyst composition of claim 74, wherein the catalytic activity of the support is less than about 1% of the catalytic activity of the Co²⁺ cations.

76. The catalyst composition of claim 74, comprising about 0.1 wt % to about 20 wt % cobalt (Co), or about 0.25 wt % to about 16 wt % Co.

77. The catalyst composition of claim 74, wherein the precious metal comprises at least one of platinum (Pt), gold, silver, copper and palladium.

78. The catalyst composition of claim 74, wherein the support comprises a surface density of about 0.1 Co atoms/nm² to about 20 Co atoms/nm².

79. The catalyst composition of claim 74, wherein the support comprises at least one of silicon dioxide (SiO₂), aluminum oxide (Al₂O₃), fumed Al₂O₃, aluminum oxyhydroxide (AlOₓ(OH)₃₋₂ₓ) and silicon oxyhydroxide (SiOₓ(OH)₂₋ₓ).

80. The catalyst composition of claim 74, wherein the support comprises at least one of gamma-Al₂O₃, delta-Al₂O₃ and theta-Al₂O₃.

81. The catalyst composition of claim 74, comprising a BET surface area of about 1 m² g⁻¹ to about 400 m² g⁻¹.

82. The catalyst composition of claim 74, wherein the catalyst composition comprises a plurality of units.

83. The catalyst composition of claim 82, wherein the catalyst composition comprises a plurality of units, and wherein the plurality of units comprise at least one of particles, powder, extrudates, tablets, pellets, agglomerates and granules.

84. A method of preparing a catalyst composition, comprising:
loading cobalt (II) (Co²⁺) cations onto a support,
wherein the catalyst composition is free of at least one of chromium and a precious metal.

85. The method of claim 84, wherein the loading comprises at least one of grafting, doping, co-precipitating and impregnating the Co²⁺ cations onto the support.

86. The method of claim 84, wherein the loading comprises contacting the support with an aqueous solution of at least one of a cobalt (II) carboxylate, a cobalt (II) glycolate and a cobalt (II) citrate.

87. The method of claim 86, wherein the cobalt (II) carboxylate comprises cobalt (II) acetate tetrahydrate.

88. The method of claim 86, wherein the aqueous solution is at a concentration of about 10 wt % up to the solubility of the at least one of cobalt (II) carboxylate, cobalt (II) glycolate and cobalt (II) citrate in the solution.

89. The method of claim 86, wherein the aqueous solution is at a concentration of about 10 wt % to about 100 wt % of the at least one of cobalt (II) carboxylate, cobalt (II) glycolate and cobalt (II) citrate by weight of the solution.

90. A method for dehydrogenating at least one of a light alkane gas and a light alkene gas, comprising:
contacting the at least one light alkane gas and light alkene gas with a catalyst composition comprising a support comprising cobalt (II) cations (Co²⁺).

91. The method of claim 90, wherein the contacting comprises combining the at least one light alkane gas or light alkene gas with hydrogen (H₂), oxygen (O₂) or carbon dioxide (CO₂) in the presence of the catalyst composition.

92. The method of claim 91, wherein the combining is at a molar ratio of H₂, O₂ or CO₂ to light alkane gas or light alkene gas of about 1:100 to about 1:1.

93. The method of claim 91, wherein the H₂, O₂ or CO₂ is present in an amount per mass of catalyst of about 1 mol kg⁻¹ h⁻¹ to about 100 mol kg⁻¹ h⁻¹, and wherein the combining with H₂, O₂ or CO₂ is at a temperature of about 500 K to about 1000 K, or about 700 K to about 900 K.
